(19) United States
(12) Patent Application Publication
(10) Pub. No.: US 2005/0165460 A1
(43) Pub. Date: Jul. 28, 2005

(54) Neuro-Electric-Therapy Headset

(76) Inventor: Onje' Erfan, Denver, CO (US)

Correspondence Address: KYLE W. ROST, 5490 AUTUMN CT., GREENWOOD VILLAGE, CO, 80111, US

(21) Appl. No.: 10/707,921

(22) Filed: Jan. 26, 2004

(51) Int. Cl. A61N 1/18

(52) U.S. Cl. 607/57

(57) ABSTRACT

A self-contained, portable headset carries a waveform source device and tissue interface circuits in a self-locating position for delivering treatment signals to a preselected area in the conch of the ear of a human subject. An electronics housing carries a waveform source device in communication with right and left tissue interface circuits, carried respectively in right and left earpiece housings. The headset carries each earpiece housing at a rearward and downward angle so that a protruding trunk enters the conch of the outer ear and contacts the conch generally below and rearwardly of the ear canal. An audio speaker delivers associated tones during treatment. An end wall of the trunk carries an array of electrodes contacts the preselected area in the conch of the ear.

### BACKGROUND OF INVENTION

1. Field of the Invention

The invention generally relates to surgery and more specifically to measuring electrical impedance or conductance of a body portion. The invention also relates to surgery and to the electrical stimulation of nerves, such as transcutaneous electrical nerve stimulation (TENS).

2. Description of the Prior Art

In the art of neuro-electric therapy, nerve endings in the auricles are stimulated to elicit physiologic and neurological responses. Certain locations in the ear or body, such as joints and muscles, may be stimulated by electrical impulses in order to produce a positive therapeutic influence on corresponding body functions, reactions, muscles, organs, systems and the like.

The applicant previously developed a neuro-electric-therapy device known as the NET-1 (a trademark of Auri-Stim Medical, Inc., of Denver, Colo.), which is one of few, if any, similar devices that have been evaluated in randomized clinical trials and clinically shown to be effective as an aid to manage headaches, addictions, and other conditions. The NET-1 is a compact, solid-state, digital device that contains a signal generator with an on-board battery. This device sends subtle electrical signals through nerve endings in the outer ear to the brain. In some cases, the NET-1 has found use in minimizing or eliminating common symptoms such as severe pain, nausea, vomiting, photo-sensitivity, sensitivity to sound and odor, blurred vision, lack of concentration, stress, depression, and other symptoms associated with migraine and hormonal migraine. This device may reduce withdrawal symptoms from use of nicotine, narcotics, and alcohol. Also, NET-1 has shown use in reducing the symptoms of menstrual disorders such as PMS and related headaches.

The NET-1 device has been described on the Internet or World Wide Web at URL address netidevice.com. This web site explains a possible basis for the successful operation and beneficial results obtained by the NET-1 device. There are 12 cranial nerves. The Vagus nerve, which is the tenth cranial nerve, extends through the neck and thorax into the abdomen. One branch of the Vagus nerve surfaces on the depression immediately behind the ear canal. This nerve is called the Arnold's branch of the Vagus nerve. Motor fibers of the Arnold's branch of the Vagus nerve innervate heart, lungs, bronchi, and gastrointestinal tract. The sensory fibers innervate, heart, lungs, bronchi, trachea, larynx, pharynx, gastrointestinal tract and external ear. This branch of the Vagus nerve serves as an access point to the brain and central nervous system allowing the treatment of certain disorders non-invasively, by neuroelectric-therapy.

All living organisms function by receiving, sending, analyzing, and responding to recognizable signals. These include, but are not necessarily limited to hormonal, chemical, audio, visual, and electrical signals. When the brain recognizes signals, it responds by secreting neurochemicals, neurohormones, neurotransmitters and other chemicals that regulate brain functions. If the brain does not recognize signals, there will be no responses, no regulation of function, and no relief from the symptoms of various disorders.

It is thought that NET-1's effectiveness comes from transmitting a set of well-defined and subtle electrical signals to the brain, inducing the brain to regulate secretion of certain biochemical agents, analgesic opiates such as beta-endorphin and enkephalin, and neurotransmitters such as serotonin, dopamine and others that are implicated in migraine, hormonal headaches, chronic pain, depression, addiction, and other disorders. One theory explaining the efficacy of this treatment comes from the mediation by NET-1 of the complex interaction of these neurochemicals, which have a wide range of physiological effects on the body that include relief from the disorders described above.

The NET-1 device is used by placing it on the outer ear at the center of depression behind the ear canal, with a built-in probe formed of concentric electrodes in contact with the surface location of the Vagus nerve. Closing a pressure switch activates operation. A typical treatment for chronic headaches, migraine headache, hormonally induced migraine (PMS), and narcotics withdrawal symptoms requires 10-15 minutes of use. The device can be used on either ear, but it is preferred to first use with the affected side and then on the other, if required.

The NET-1 has been evaluated in a double-blind placebo clinical trial for treatment of chronic migraine headache and premenstrual syndrome. According to test results, seventy-two percent of the volunteer subjects who were randomly assigned active devices reported significant reduction in the frequency and intensity of their headaches and associated symptoms. This resulted in a major reduction in their use of prescription drugs and visits to hospital emergency rooms. Forty-three percent of the subjects who were randomly assigned placebo devices reported similar effects.

Two other clinical studies indicated favorable responses ranging from thirty-five percent for active devices versus eighteen percent for the placebo. A separate study with no placebo devices showed a fifty-six percent positive response. The study design followed the guidelines established by the International Headache Society (IHS), and each study lasted for three months.

During the course of the clinical studies of nicotine addition, it was observed that neuro-electric therapy administered through the NET-1 device also was effective for treating migraine headaches. Many subjects who suffer from debilitating migraines are also dependent on narcotics for relief of their headaches. This dependency creates other medical problems and concerns for many migraine sufferers. Since the NET-1 device is also effective for addiction therapy, the user can treat both conditions at the same time.

The effectiveness of NET-1 for smoking cessation was evaluated in ten clinical trials conducted at three different sites with volunteer subjects. The NET-1 was found to be sixty percent effective in treating cigarette addiction compared to fifteen percent effectiveness using a placebo NET-1 device, without the use of adjunctive therapy. These and other randomized, double blind, placebo, clinical trials are believed to be unique in this area of study. These trials have established neurological and physiologic response by Arnold's branch of the Vagus nerve.

While the NET-1 device has proven effectiveness, its proper use requires that each patient should be trained in applying and positioning the electrical interface to the individual patient's ear and in its proper operation. The need for introductory training seriously limits NET-1 from wide distribution and use. Prospective users in need of treatment may be reluctant to obtain training, whether because of cost, shortage of time, or lack of available access. Further, a device such as NET-1 is practical for use only if the patient is able to self-administer. This result is inherent with the type of conditions being treated, which can arise suddenly and frequently.

It would be desirable to create a device and method of treatment having the proven effectiveness of NET-1 combined with automatic self-positioning of the electrical interface.

Further, it would be desirable to create a treating device and method of treatment that can be used without an introductory training session. A prospective patient should be able to self-apply and self-administer effective treatments with minimal instruction and without knowledge of auricular medicine.

Recently developed technology employs electrical signals to diagnose and treat disparate conditions. For example, transcutaneous electrical nerve stimulation (TENS) is now practiced in many schools of treatment or diagnosis. Medical instruments are known for applying electrical energy in selected waveforms to the human body. U.S. Pat. No. 3,894,532 to Morey shows an instrument for generating electrical pulses in a sine wave of variable frequency to a selected point on the human body. Similar to TENS devices, the Morey instrument requires that a patient hold a hand-held electrode, while a physician-administrator applies a circuit-completing electrode to sensitive spots on the patient's skin. Such usage of separated electrodes is common in TENS machines and apparently is related to the need to locate specific points on the patient's body. Quite clearly, suitable administration of such a device requires specialized knowledge of treatment points and likely requires the help of a trained administrator to handle the equipment.

Combined techniques of acupuncture and auricular medicine are known from recent U.S. Pat. No. 5,514,175 to Kim. A trained administrator such as a physician must apply an impedance measuring device to custom-locate multiple treatment points, in this case identified as acupuncture points, on the patient's ear. Then, a custom-structured contact assembly is formed to hold output contacts on the custom-located points. As with a TENS machine, a remote circuit-closing electrode is applied to a location external from the ear, such as to the mastoid process. The same device and treatment can be applied to both hears by connecting two similar devices on one headband, and both can be operated from a common signal source by electrical interconnection. As known from prior methods, Kim states that a physician must locate the necessary points and fit the custom-structured contact assembly, after which a layperson might be able to administer subsequent treatment. This patent teaches a critical importance in locating acupuncture contact points, suggesting that the locations of acupuncture points are not sufficiently predictable that the customized steps could be eliminated for many patients.

Other patents show additional treatment of areas near the ear by various equipment. U.S. Pat. No. 4,112,923 to Tomecek shows a probe for applying electricity at a selected frequency to the human body, including the ear, via a concentric probe. U.S. Pat. No. 4,503,863 to Katims shows an electrical stimulator that has its opposite electrodes attached to opposite sides of a patient's jaw, just in front of the earlobe, and for passing current between them. A portion of the current is for stimulating an auricular branch of the Vagus nerve. Three waveform generators are for supplying selected frequencies and combinations of frequencies. U.S. Pat. No. 4,690,145 to King-Smith discloses a microprocessor for storing digital waveforms and a D-A converter for generating an applied analog waveform. This arrangement is intended to provide a wide variety of signals. U.S. Pat. No. 5,458,625 to Kendall shows a device for stimulating nerves, especially for stimulating the Vagus nerve by attaching electrode clips to both ears. Each clip carries two closely spaced electrode pads that are spaced for avoiding short circuits and for avoiding trans-cranial currents. The pads on each clip are connected to a signal-generating device for supplying electrical energy through the pair of pads on the clip. The pads are for making sufficient contact with springs of the Vagus nerve on each ear to produce a desired stimulation. The stimulation is described as useful for treating withdrawal from addictions, relief of pain, and relief of stress.

Although this variety of devices and methods are known previously, each requires a substantial base of knowledge in order to apply and use the device or method. The nature of the equipment and of the treatment shows a need for a specialist to assist in administering the device and the treatment.

As mentioned, above, the devices described in this array of patent art often recite that they achieve certain beneficial treatment results. However, the patent specifications are notably devoid of reference to any clinical studies supporting the recited treatment results. Consequently, a degree of skepticism may be warranted before such treatment results and peripheral observations may deserve full accreditation.

These examples from the prior art show that devices have been designed for generating waveforms of many varied types. The devices vary frequency, amplitude, and other characteristics of the waveform. Both digital and analog waveforms have been created, can be stored for replication, and can be converted between digital and analog modes. However, as best shown by the most recent patent to Kendall, the devices still can be large and complex to administer. Smaller devices such as NET-1 are known, but they are limited in their ability to provide coordinated stimulation at both ears.

It would be desirable to improve the performance of nerve stimulating devices in order to provide more effective, proven treatments. In particular, it would be desirable to create an improved portable treatment device that the patient can carry and apply to himself, both for introductory usage and for subsequent usage. Such an effective, portable, and self-applied device enables the patient to treat himself at unpredictable, critical times when his condition recurs. Thus, for example, a headache or a craving for nicotine or some other drug can recur without warning. Obtaining prompt relief is highly important. The most critical factor, missing from prior art, is a reliably accurate apparatus structure, system, and method for self-locating the electrical interface on an area of the ear that is suitable to receive such treatment.

To achieve the foregoing and other objects and in accordance with the purpose of the present invention, as embodied and broadly described herein, the auricular stimulator of the invention may comprise the following.

### SUMMARY OF INVENTION

Against the described background, it is therefore a general object of the invention to provide an improved auricular stimulator employing the effective therapy established by NET-1, for the treatment of smoking and other addictions or drug-related disorders, headaches, pain control, and the like, wherein the device is automatically self-locating at an area of the ear that is an effective site for receiving treatment.

Yet another object and advantage of the invention is to provide an improved device, as compared to NET-1, that may be used by a person without prior personal instruction, enabling the person to control various addictive behavior or physiologic disorders as they arise.

Another object is to comprehensively treat different disorders that may respond preferentially to therapy on different sides of the cranium.

Additional objects, advantages and novel features of the invention shall be set forth in part in the description that follows, and in part will become apparent to those skilled in the art upon examination of the following or may be learned by the practice of the invention. The object and the advantages of the invention may be realized and attained by means of the instrumentalities and in combinations particularly pointed out in the appended claims.

According to the invention, a self-administrable and self-locating neuro-electric-therapy headset carries a waveform source device and delivers treatment signals to an effective treatment area in the ear of a human subject, who typically will be in need of treatment. The headset is structured to automatically applying tissue interface circuits for delivering treatment signals to a preselected contact area in the conch of each ear of the human subject. An electronics housing carries a waveform source device having an impedance detecting function. Right and left earpiece housings are each connected to the electronics housing and are carried in suitable positions for application, respectively, to the right and left ears of a human subject. The right and left earpiece housings include right and a left elongated protrusions, each extending to a respective free end wall from the right and left earpiece housings. The earpiece housings carry a tissue interface circuit on the free end wall, and the tissue interface circuits are in communication with the waveform source device for communicating impedance and receiving treatment signals. Also, the elongated protrusions are suitably arranged for applying the respective tissue interface circuits against the conch of the human ear when the headset is applied to a human subject. The preselected contact area is juxtaposed to the lower edge of the ear canal opening and extends rearwardly in the conch of the ear. The tissue interface circuit comprises an array of electrodes carried in association with the free end wall of each earpiece housing and is sized to typically contact at least about one-quarter the height of the conch of a human ear. The array is arranged to achieve electrical communication with the preselected contact areas.

Another aspect of the invention is a self-administered method of treating disorders such as chronic headaches, migraine headache, hormonally induced migraine (PMS), and narcotics withdrawal symptoms. An effective treatment delivers neuro-electric-therapy to a human subject suffering the disorder and in need of such treatment. The method is performed by use of a self-contained, portable headset that carries a selectively activated waveform source device that also measures impedance and causes an audible signal responsive to the measured impedance. Right and left earpiece housings each carry a tissue interface circuit that is responsive to the source device to deliver waveforms and to measure impedance. The tissue interface circuit is configured with an ear-entering portion that is suitably sized and shaped for application onto the conch of the human ear. The ear-entering portion has a free end that carries a contact portion of the tissue interface circuit. The contact portion is an array of electrodes suitably sized and shaped for contacting the conch of a human ear and, specifically, for contacting a preselected contact area near the lower edge of the ear canal opening and extending rearwardly from the canal. The interface circuits are applied to the ears of the human subject, in a position such that the tissue interface circuit is in electrical communication with the preselected contact area. The source device is activated to provide electrical output signals to the tissue interface circuits while simultaneously measuring impedance at the tissue interface circuits and generating an audible signal. As a result, the tissue interface circuits deliver an effective waveform treatment over a time period effective for treatment, while simultaneously generating and delivering an audible signal responsive to impedance at the tissue interface circuits for enabling the human subject to adjust the position of the headset for electrical communication with the preselected contact area.

The accompanying drawings, which are incorporated in and form a part of the specification illustrate preferred embodiments of the present invention, and together with the description, serve to explain the principles of the invention. In the drawings:

### BRIEF DESCRIPTION OF DRAWINGS

FIG. 1 is a front elevational view of a neuro-electric-therapy headset.

FIG. 2 is a rear elevational view thereof.

FIG. 3 is an exploded partial view of a tissue interface circuit in one earpiece housing of the headset.

FIG. 4 is an isometric partial view of the tissue interface circuit of FIG. 3 in assembled condition in one earpiece housing.

FIG. 5 is an isometric view of the headset band with an earpiece housing shown in exploded view near its attachment point to the band.

FIG. 6 is an isometric view of the headset with control module shown in exploded view.

FIG. 7 is a schematic side view of the outer structure of a human ear showing an effective treatment area for application of the headset in the conch of the ear.

### DETAILED DESCRIPTION

The invention is a neuro-electric-therapy headset 10 that simultaneously provides coordinated electrical stimulation to a preselected area in the conch of a human ear, for example to the Arnold's branch of the Vagus nerve, at both ears of a patient. The headset configuration enables self-administration of treatment by substantially anyone. The design of the headset automatically locates a pair of tissue interface circuits in proper proximity to administer treatment to the human ear, such as to areas known to stimulate the hypothalamus or to Arnold's branch of the Vagus nerve. The headset provides the automatic location function by suitably carrying the tissue interface circuits, typically at a rearward and downward angle relative to the normal wearing position of the headset. In addition, the tissue interface circuits are carried at the ends of guiding protrusions that follow the contours of the ear to enter the conch area. Finally, the area of each tissue interface circuit is sufficiently sized for at least some of the electrodes forming each circuit to achieve treating contact with the preselected treatment area. Thus, the headset 10 provides an improved and easily operated vehicle for administering bilateral neuro-electric-therapy.

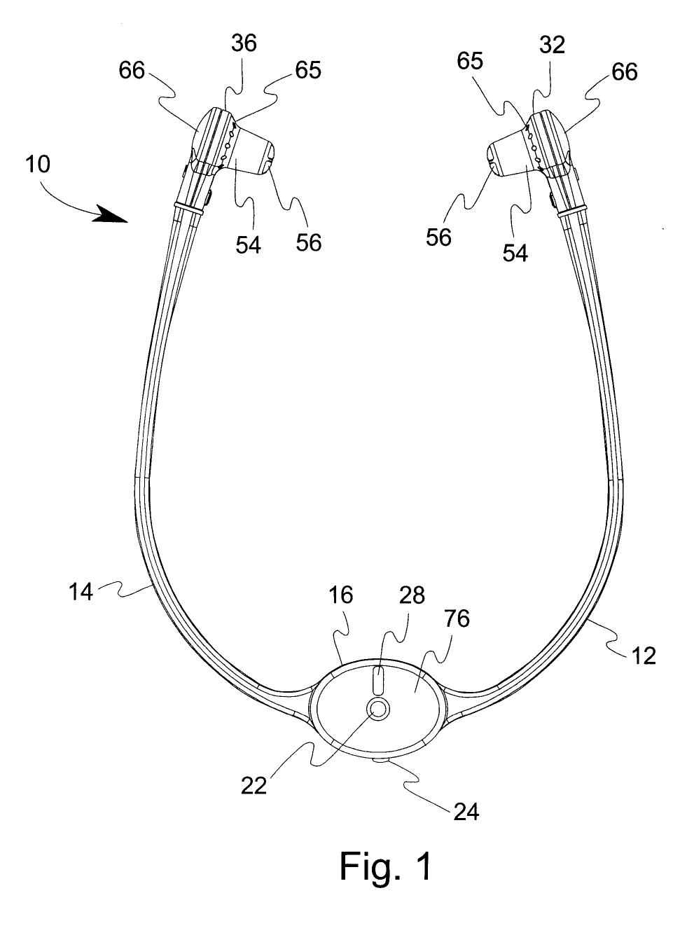

With reference to FIGS. 1-6, the headset 10 provides a head-engaging band or bow, which is composed of a discrete left bow arm 12 and a right bow arm 14. The headset preferably is applied and worn similar to a stethoscope, around the bottom of the head rather than over the top. The headset 10 includes an electronics housing 16, which preferably is carried near the center of the bow, between the left and right bow arms. Housing 16 provides an electronic control module that is a source of selected electrical outputs or a means for supplying electrical stimulation. The prior art contains numerous detailed descriptions of apparatus for supplying electrical stimulation. Those patents mentioned in the discussion of background art, specifically U.S. Pat. Nos. 3,894,532, 4,112,923, 4,503,863, 4,690,145, and 5,458,625, are incorporated by reference for teaching the possible construction and operation of suitable signal generators or other source means or devices. The preferred source of electrical stimulation is a solid state, advanced technology, digital device, powered by one or more on-board batteries. Self-contained, single ear source means or devices of this small size already are known and commercially available, such as in the NET-1 device produced by this inventor's company.

In addition, the electronic control module provides a self-diagnostic circuit that identifies low impedance points in the ear. Because the headset is configured to be self-administered and self-locating of the desired treatment area, the self-diagnostic circuit provides an audible signal to show that points of suitable low impedance are in contact with the tissue interface circuits. Consequently, the user can slightly reposition the headset as required to obtain a confirming signal. The tissue interface circuit on each earpiece provides a large area array of electrodes so that at least some of the electrodes will achieve contact with the suitable treatment area, thereby typically avoiding any need to reposition the headset. The large area array typically will cover about one quarter to one half the height of the conch, so that suitable contact is almost unavoidable. The large area array is carried in a predetermined position or range of positions, generally in a downward angular position and optionally also in a rearward angular position with respect to the headset so that automatic application to the conch, near the lower edge of the ear canal and rearward there from, is almost unavoidable. Thus, the headset is suited for self-administration and is self-locating over a suitable contact area.

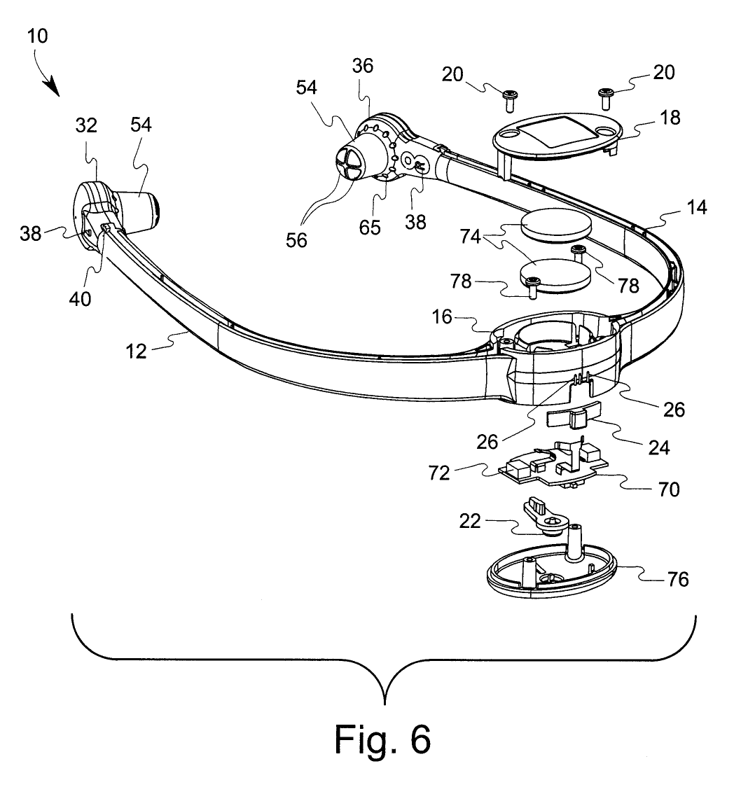

In order to receive the source means or device, the housing 16 includes a removable back cover 18 on the rear face of the headset. Removable screws 20 attach the cover. The front, opposite cover or face of the housing carries a selectively actuated means such as an on-off push button switch 22 that communicates with the source device. As best shown in FIG. 6, the bottom edge of the housing can carry a mode selection switch 24. The view of FIG. 6 shows that the selection switch is provided with two position indicia 26 marked on housing 16 as positions 11 and 1. The switch 24 readily could have had three or more positions, if desired. Mode switch 24 communicates with the source device for switching between two or more modes of operation, which may include but are not limited to switching between two signal levels, two waveform sets, or preferably between two levels of electrical output.

A light window 28 shares the front wall with switch 22. The housing may carry one or more LEDs or other indicator means in the window 28. An LED in this window may indicate one or more desired properties of operation. By way of example, these may include on-off status, mode of operation, intensity of signal, frequency, time of session, or other parameter.

The left bow arm 12 and right bow arm 14 extend from each side of the housing 16 to a free end. Each bow arm is elongated between the housing 16 and the free end. Each free end carries a respective earpiece housing. The earpiece housings each carry a tissue interface circuit that is in electrical communication with the source device in housing 16. The means for communicating may be any known type, such as conductors in or associated with the bow arms. For this purpose, the bow arms may have suitable conductors, passages, or slots so that they carry signals or signal wires from the electronics housing 16. Alternatively, wireless communications may be used to convey the desired signals from the source device to each tissue interface circuit.

The two bow arms are spaced apart at their free ends so that the headset defines a central area sized to receive the chin or lower face of a user. The left bow arm 12 terminates at a left free end, which is configured as a pivot ring 30 for receiving and pivotally carrying a left earpiece housing 32. The right bow arm 14 similarly terminates in a right free end configured as a pivot ring 34 for receiving and carrying an associated right earpiece housing 36 by a pivotable connection. As best shown in FIG. 6, the earpiece housings carry indicia 38, such as R or L, to designate whether each is a right side or left side earpiece housing. These indicia 38 aid to the wearer in properly orienting and applying the headset. The indicia 38 appear on both front and back portions of the earpiece housings, to ensure that each earpiece is assembled correctly and placed on the correct bow arm during assembly. Proper assembly ensures that each earpiece will pivot suitably and properly contact the user's ear, as explained below.

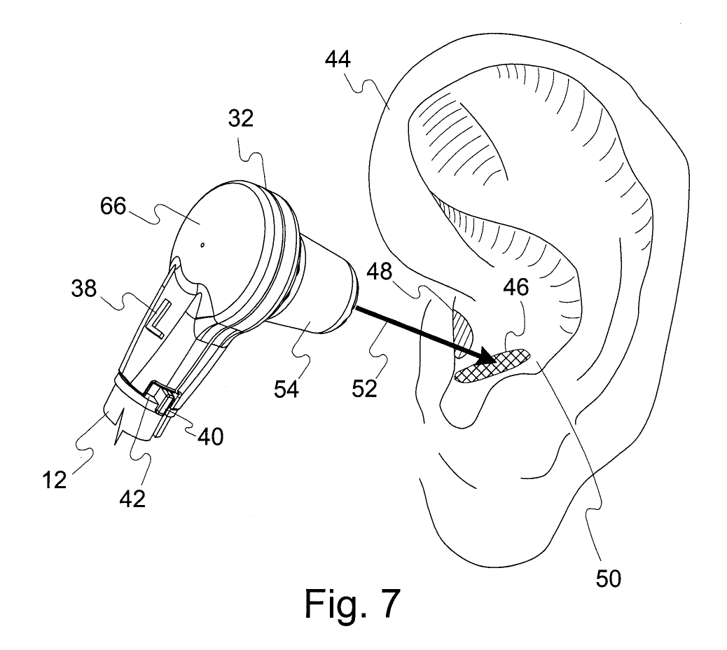

Each earpiece housing is pivotable on a respective pivot means such as a pivot ring 30, 34, or like structure for enabling pivotal rotation on a longitudinal axis of the associated bow arm to suitably orient the earpiece housing to enter the conch of a human ear and contact an effective treatment area. According to a preferred structure, only a limited degree of motion is permitted so that the earpiece housings will be self-aligning with the desired contact area of an ear. A pivot limiting means such as a lug 40 on the end of each right and left bow arm engages in a mating gap, slot, or groove 42 in the earpiece housing for restricting pivotal motion, as best shown in FIG. 7. The matching earpiece housing receives the lug 40 in the mating gap, slot, or groove 42, which may be located at the parting line between separable portions of the earpiece housing. The groove provides clearance over a limited arc of rotation for the earpiece housing with respect to lug 40. Thus, each earpiece housing 32, 36 is permitted to pivot or rotate through a narrowly defined acute angle. The limited pivotal motion is an optional feature, desired for best application and control. Alternatively, the angular positions of the earpiece housings may be fixed. Pivotal motion aids the application and fit of the tissue interface circuits to an effective treatment area in the conch of an ear. Pivotal motion in both front and rear directions can be acceptable in some situations.

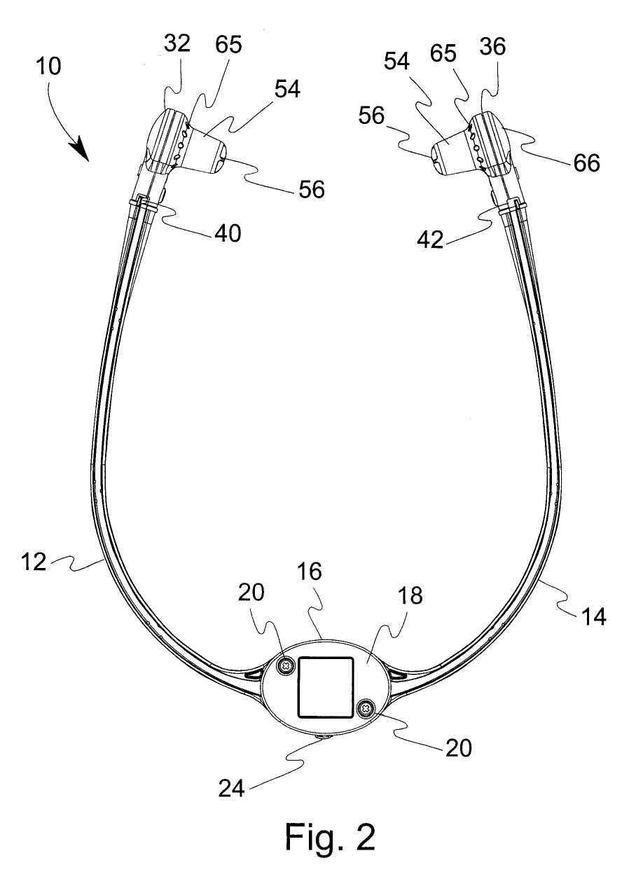

Typically, the earpiece housings rotate through a preselected range that may include as one limit a neutral position or zero angle, as suggested in FIGS. 1 and 2, wherein the earpiece housings face each other or lie in the plane of the headpiece. The range may include an opposite or rearward angle limit wherein the earpiece housings are angled toward the rear face of the headset by a preselected angle from the neutral position. As an example, twenty to thirty degrees may be a suitable preselected limit of the rearward angle. When the headset is applied for wearing, the earpiece housings both can be pivoted within the preselected range to best align for entering the conch of the ear. The permitted angle, to only one side of neutral, ensures that the headset will tend to be worn with proper right-to-left orientation. Wearing the preferred headset with backwards orientation does not readily, conveniently, or comfortably allow the earpieces to enter the conch of the ear in many instances.

FIG. 7 shows the relationship between the outer structure of a typical human ear 44 and the desired treatment area 46 of the ear. The ear canal 48 serves as an easily located reference point. Conch 50 is the depressed area of the outer ear structure immediately behind the ear canal 48. The drawing schematically illustrates the area 46 of the conch where stimulation is best applied. In auricular medicine, contact area 46 is known to lie along areas for stimulating the anterior hypothalamus, the hypothalamus, and the posterior hypothalamus. These points or areas generally lie in the conch of the ear, below and behind the ear canal opening, with a slight upward trend rearwardly from the ear canal. This area also corresponds to an area where Arnold's branch of the Vagus nerve can be suitably electrically stimulated. Thus, the conch 50 is a general target for the earpiece housings 32, 36. A more specific target is the lower portion of the conch, rearwardly from the lower edge of the ear canal. Arrow 52 suggests the preferred approach for an earpiece housing 32 to enter conch 50 with a slight rearward angle in order to deliver the tissue interface circuit to an effective treatment location.

The earpiece housings are structured to enter the conch 50 and deliver the tissue interface circuits into electrical contact with treatment location 46. Each earpiece housing is formed of two sub-parts. A front sub-part of the earpiece housing defines a protrusion 54 that is generally formed as a frusto-conical trunk of suitable length, width, and shape to enter the conch area of the ear. Each protrusion extends approximately normal to its bow arm. The protrusion 54 carries a tip that defines an exposed, free end of the earpiece. The tip is smoothly shaped in a convex contour that corresponds to the approximate contour of a concave inner surface of a typical conch.

FIG. 6 shows the free end of protrusion 54, which provides a framework to carry and support waveform delivery devices for administering treatments. The waveform delivery devices substantially fully occupy the free end as shown in the drawings. The waveform delivery devices define a tissue interface circuit that includes a large area array of contact electrodes 56. The electrodes occupy substantially the entire free end of the protrusions 54. Suitable electrodes are formed of conductive metal such as copper, gold, silver, or the like. Together, the electrodes may form a circular array having a diameter related to the typical dimensions of the conch of a human ear. The diameter is about 6.4 mm to 9.5 mm (0.25 to 0.375 inches), which is about one quarter to one half the height of the conch. This dimension is suitable to ensure a high likelihood of contact with treatment area 46, when the headset 10 is worn from the bottom of the head. The earpieces typically are worn with the protrusions 54 angling slightly downwardly, to contact the lower part of the conch of the ear, which further creates a likelihood of contacting the treatment area 46. The downward angle of the protrusions 54 is a result of resiliency in the bow arms 12, 14, such that the bow arms close against the head beyond a vertical or mutually parallel position. In FIG. 7, the disposition of arrow 52 suggests such a downward angle. Of course, a headpiece 10 could be designed to achieve the desired positioning of the electrodes even if worn over the top of the head.

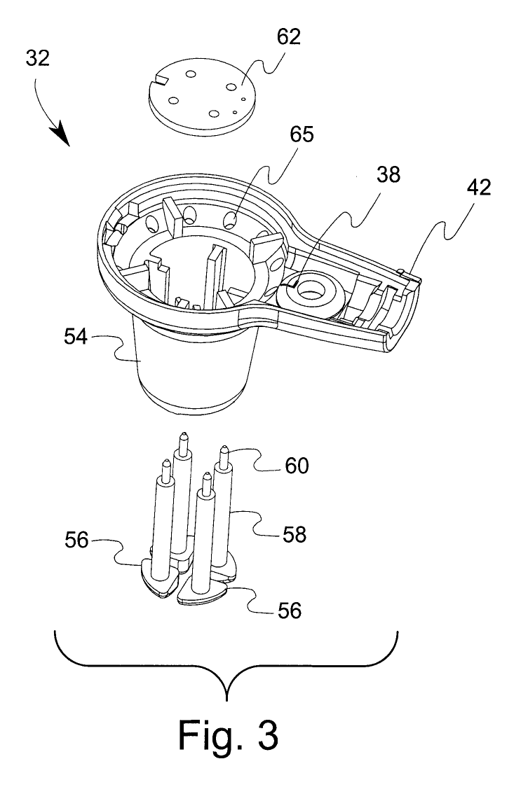

FIG. 3 best shows the arrangement and structure of electrodes 56. Each tissue interface circuit preferably includes four contact electrodes 56 having quadrant-shaped outer ends, each generally configured as a quadrant of a circle. A shank 58 extends rearwardly from the backside of each electrode outer end 56 and through the length of earpiece housing trunk 54. A narrower portion 60 at the rear end of each shank 58 is sized to engage and mount through a dielectric spacer board 62, which secures and aligns the electrodes within the earpiece housing. The spacer board 62 fits within the earpiece housing in a predetermined orientation, engaged with an alignment notch of the housing so that the electrodes are maintained in alignment between the spacer and the contact face of the earpiece housing. The spacer board 62 may be a printed circuit board and may carry electrical components such as a receiver, if wireless communication to the earpiece is employed, or a signal source. Connecting tips 60 serve as interfaces for receiving waveform or other signals. Tips 60 can be electrically connected to circuitry either on spacer board 62 or on the signal source in housing 16, through the bow arms. Thus, the tips 60 are connectable to the signal source by any suitable means. For example, tips 60 may extend as wire conductors through a bow arm to the signal source.

The electrodes 56 protrude from the free end of trunk 54 as a contact face of the tissue interface circuit for achieving reliable electrical contact with a suitable area of the conch of a user's ear. Preferably, the electrodes 56 are mounted in apertures formed in the end wall of trunk 54. If desired, the electrodes can be resiliently carried, such as on springs or resilient supports.

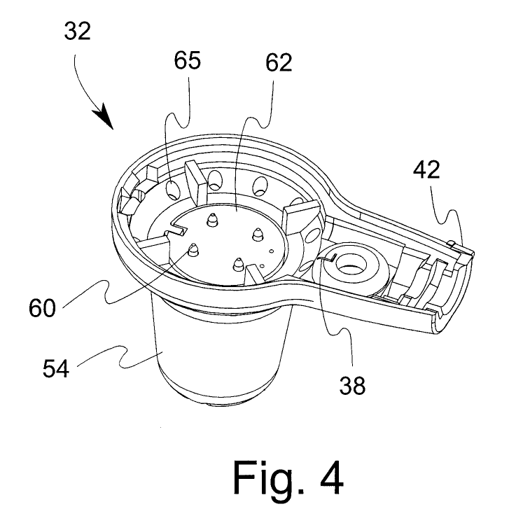

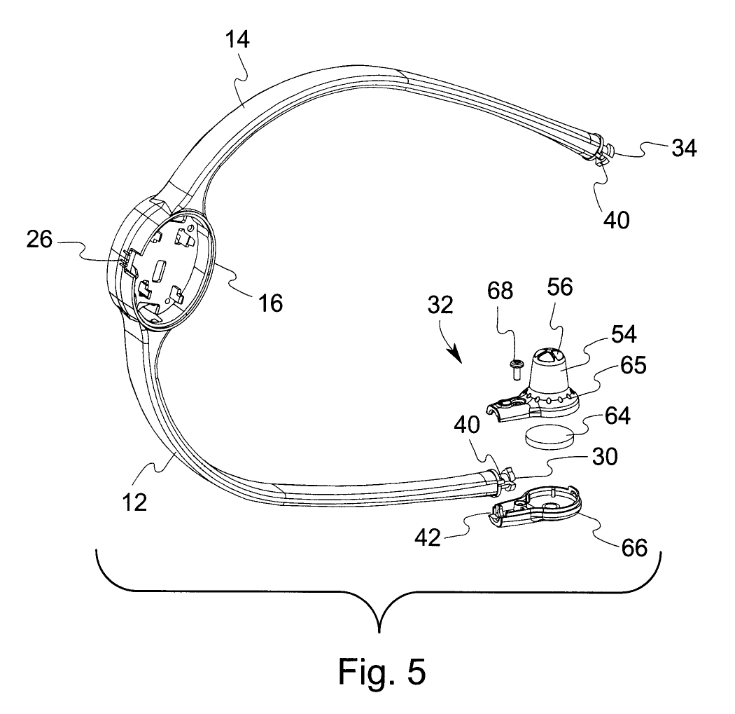

As shown in FIG. 5, a speaker 64 is positioned against the backside of the dielectric spacer 62. The speaker is connected to the waveform source device to provide constant audible signals as well as additional periodic audible signals when the headset is in use delivering electrical signals through the tissue interface circuit. With respect to the periodic audible signals, the waveform source device may provide a thirty second reset of the speaker. The earpiece housings 32, 36 provide an annular array of sound passages 65 surrounding the base of trunk 54 for delivering the audible signals to the user. As best shown in FIG. 4, the array of sound passages 65 is located behind the dielectric spacer 62. This arrangement places the speaker 64 in uninterrupted communication with the sound passages. In turn, the sound passages are arranged around the base of protrusion 54 for efficiently directing the speaker output sound to the user's ear.

The audible signals serve multiple functions. The signals confirm to the user or human subject that the headset is in operation. This is beneficial because the waveform output through the tissue interface circuit usually is not physically detectable to the user. The audible signal confirms that treatment is in process and that the headset is working. The lack of audible signal could mean, for example, that batteries are due for replacement. The signal is adapted to confirm that the tissue interface circuits are in contact with an area having suitably low impedance. For example, the signal may assume a special tone or be silent if impedance is not suitably low. The periodic signal also aids the user in knowing the progress and termination of treatment. An effective treatment session typically extends for twenty minutes, followed by automatic shut-off. When the time period has finished, the periodic audible signal also ceases, or a termination signal can be given. In either case, the user can properly deduce when a treatment session is complete.

In some cases the audible signal may provide an improved therapeutic result. For example, the user may associate the audible signal with the waveform treatments through the tissue interface circuit. In this instance, the audible signal may provide a reinforcement or surrogate source of therapy. For this purpose, the audible signal should be delivered with sufficient frequency that it maintains association with progress of the treatment. A continuously delivered audible signal is suitable, either at a single frequency or varied frequencies. A periodic signal is suitable if delivered sufficiently often to be associated with progress of the treatment.

The earpiece housing includes a backside housing portion 66 that closes the earpiece behind the protrusion 54. A screw 68 secures the backside portion 66 in place, correspondingly holding speaker 64 in place behind dielectric board 62. The screw closes the earpiece housing on one of the pivot rings 30, 34 to enable the earpiece housing to be pivotally mounted on a bow arm 12, 14. As best shown in FIG. 6, the quadrant electrodes 56 are spaced apart on the end of trunk 54 so that they do not directly contact each other to form a short circuit. They are arranged over the majority of the surface area on the end wall of trunk 54 to contact a substantial portion of the conch 50. This arrangement ensures electrical contact preselected areas of the ear, which may include hypothalamus areas of the ear and Arnold's branch of the Vagus nerve.

The electronics housing 16 carries a waveform source device, previously mentioned. Such a device may be composed of a circuit board 70 equipped with suitable solid state electronic components to provide functions such as memory, clock timer, and microprocessor to enable the desired modes of treatment, output signals and other variable functions. The desired treatment modes are those known to be effective for the NET-1 device, which are clinically tested and proven. The circuit board carries connectors 72 for attachment to the tissue interface circuit. One or more batteries 74 provide power to the source device. A front cover 76 closes the housing 16. Internal screws 78 hold the front cover in place.

In use, the described headset is self-contained. It carries all electronics and administration equipment for providing treatment. The headset 10 applies electrical output energy from a source 70 in housing 16 through bow arms 12, 14 to the tissue interface circuits and to electrodes 56 carried on right and left earpieces 32, 36. There, the tissue interface circuit delivers treatment by electrical contact with preselected treatments areas in the conch 50 of the ear, such as hypothalamus points or Arnold's branch of the Vagus nerve.

Treatment can be administered by providing a waveform signal from the tissue interface circuit to the outer ear, especially to the conch 50, where the signal reaches preselected treatment areas that may be identified as hypothalamus areas or Arnold's branch of the Vagus nerve. Treatment may be either ipsilateral or bilateral. At the signal strengths used in the headset, best performance employs a closed circuit pathway. For example, the waveform can be considered a positive signal through selected electrodes 56 of the tissue interface circuit. Other selected electrodes of the tissue interface circuit serve as a return electrical path. For convenience, the return path may be considered to be negative. Thus, the electrodes provide a closed circuit through the preselected areas of the ear. The treated areas are in intermediate position between the positive and negative electrodes. This administration method, or any other administration method, allows all electrodes to serve in the generic capacity of signal delivery devices, whether momentarily serving in positive or negative capacity.

Each tissue interface circuit individually can apply therapy in a variety of treatment modes. Ipsilateral treatments can be applied between any selected electrodes of a single array, such as between side-by-side electrodes or between diagonal electrodes on each individual tissue interface circuit. An array 56 may be divided into two neighboring electrode pairs for applying treatment signals between halves of the array. Further, the treatments may be applied between one electrode and three electrodes of a single array. Treatments applied between electrodes of a single tissue interface circuit rely on at least some electrodes being suitably located to electrically communicate with or through the hypothalamus areas or Arnold's branch of the Vagus nerve.

The four electrodes 56 on a single contact face occupy substantially the entire contact face of protrusion 54. This large proportion of coverage creates a high probability that at least some electrodes will be suitably located in communication with hypothalamus points or Arnold's branch of the Vagus nerve. The earpiece is sized to fit a broad variety of ear sizes. The contact face may have an approximate diameter of 9.5 mm (0.375 inch) so that it will fit substantially any human ear, while the electrode array contacts a large portion of the conch near the lower edge of the ear canal and rearwardly from it.

If bilateral treatment is desired, the waveform source can provide a signal arrangement such that the electrodes of one tissue interface circuit apply a positive treatment signal and the electrodes in the other tissue interface circuit serve as negative. The signal then is transmitted transcranially between electrodes of the opposite tissue interface circuits. The quadrant electrodes allow complex variations and patterns of treatment, including mixed transmissions among electrodes of a single tissue interface circuit and trans-cranial transmissions to some or all electrodes of the opposite tissue interface circuit. The headset 10 is able to coordinate treatments between any combination of electrodes on the same tissue interface circuit or between opposite tissue interface circuits.

Because both ears are contacted at the same time, treatments to each side can be simultaneous or alternate. Thus, the headset provides a vehicle for improved versatility and efficiency in applying treatments, with an expected increase in effectiveness.

The following example illustrates a preferred method of treatment employing the headset 10.

Example 1A headset 10 is equipped with a signal source device employing the clinically proven signal modes known for use with the NET-1 device of the prior art.

For use, the headset is prepared by confirming the proper right and left side orientation, as required. Checking the indicia 38 on one or both earpiece housings confirms the orientation. Alternatively, checking the pivot direction of a earpiece housing will establish a proper orientation, as an earpiece housing pivots only to the rear, from a neutral, side-facing position.

The right and left earpiece housings are respectively placed on the right and left outer ear structures, on the conch at the center of depression behind the ear canal. Both tissue interface circuits, through the electrode arrays, are placed in communication with preselected areas of the conch known as hypothalamus points or Arnold's branch of the Vagus nerve in the respective ears.

The actuation switch 22 initiates a timed treatment period, such as twenty minutes. The switch can be actuated either before or after applying the headset. An LED light 28 confirms operation, and the signal source causes both a constant audible signal and a periodic audible beep from speakers 64, which further confirm operation. An output mode switch 24 is set to a first position for a normal electrical output level through the tissue interface circuit. A second mode position is available for delivering a higher electrical output, such as double the first position output. The choice of output level is empirical. Some users appear to need for higher output level, perhaps for individual physiological reasons.

An effective timed treatment period for chronic headaches, migraine headache, hormonally induced migraine (PMS), and narcotics withdrawal symptoms is approximately fifteen to twenty minutes of use. Throughout this period, the treatment is applied at both ears.

In a preferred treatment mode, the four electrodes 56 of each tissue interface circuit are grouped into two pairs of two each. One pair within each tissue interface circuit, which will be referred to as the positive pair, provides a signal output. The other pair of each tissue interface circuit, which will be referred to as the negative pair, provides a return electrical pathway to complete a circuit. The output signal from the positive pair of each tissue interface circuit can provide both bilateral and ipsilateral treatment. The signal can travel a completed circuit either through the negative pair of the same tissue interface circuit or through the negative pair of the opposite tissue interface circuit. The signal provides treatment both over the highly localized area juxtaposed to a single tissue interface circuit and over the broad area between and surrounding the opposite tissue interface circuits.

The treatment consists of a full spectrum of treating frequencies applied with each cycle, such one complete cycle of frequencies per second. The full spectrum includes a low range from about two to four hertz, a midrange from about fifty to eighty hertz, and a high range from about one thousand to two thousand hertz. The full spectrum is delivered in order to provide a broad range of therapy and to enable the user to self-administer the treatments without the needless complication of selecting specific frequencies. Those frequencies within the indicated spectrum that are effective will be therapeutic, while any others are harmless.

The simultaneous treatment of both left and right sides is beneficial. Certain conditions frequently respond better to treatment on a specific side. Numerous trails have shown that treatment on the right side tends to be preferred in many human subjects for treating conditions including headache, PMS, smoking cessation, and narcotics withdrawal symptoms. Treatment on the left side tends to be preferred in many human subjects to relieve depression, stress, and anxiety. Notably, depression, stress, and anxiety are frequent side effects of pain. Consequently, a treatment simultaneously delivered to both left and right sides is well suited to provide relief for both a painful condition such as headache and the depression that frequently accompanies the painful condition.

The treatment automatically proceeds for a predetermined time period that has been found effective for treatment. A suitable time period is between fifteen and twenty minutes. At thirty second intervals during the treatment period, the signal source causes a pulsed tone or beep to issue through the speakers. At the conclusion of the timed period, the signal source automatically ceases operation and the headset shuts-off.

The headset 10 operates in a treatment mode meeting the following specifications:

TABLE 1
Treatment Modes

| TREATMENT | |
|---|---|
| MODE | |
| Output Voltage | 0 to plus/minus 6 v. max. probe to ring |
| Output Current | 0 to plus/minus 86 μamps probe to ring |
| Output Waveform | Complex series of square variable waveforms |
| Output | 5 simultaneous fundamentals with harmonics. Three |
| Frequencies | simultaneous fundamentals into audio transducer with |
| | additional feedback tones. Preferred frequencies |
| | are 2.5, 5, 10, 20, 40, 80, l46, 160 Hz |
| Audible | Pulsing tone at a predetermined pulse modulated by |
| Detection | quality of the point |
| Timer | Beeps every 30 seconds |
| Power | Two replaceable 3 v. lithium cells. Battery life |
| | approximately 8,000 minutes of continuous use |
| Operating | 20-120 degrees F. (−7.5-48.5 degrees C.) |
| Temperature | |

The foregoing is considered as illustrative only of the principles of the invention. Further, since numerous modifications and changes will readily occur to those skilled in the art, it is not desired to limit the invention to the exact construction and operation shown and described. Accordingly, all suitable modifications and equivalents may be regarded as falling within the scope of the invention as defined by the claims that follow.

1. A self-administrable and self-locating neuro-electric-therapy headset for automatically applying tissue interface circuits for delivering treatment signals to a preselected contact area in the conch of each ear of a human subject, comprising:
an electronics housing carrying a waveform source device having an impedance detecting function;
right and left earpiece housings each connected to said electronics housing and carried in suitable positions for application, respectively, to the right and left ears of a human subject;
said right and left earpiece housings further comprising a right and a left elongated protrusion, each extending to a respective free end wall, respectively from said right and left earpiece housings, and each carrying a tissue interface circuit on said free end wall, wherein said tissue interface circuits are in communication with said waveform source device for communicating impedance and receiving treatment signals, and said elongated protrusions are suitably arranged for applying the respective tissue interface circuits against the conch of a human ear when the headset is applied to a human subject, over a preselected contact area juxtaposed to the lower edge of the ear canal opening and extending rearward there from in the conch of the ear;
wherein said tissue interface circuit comprises an array of electrodes carried in association with said free end wall of each earpiece housing, sized to typically contact at least about one-quarter the height of the conch of a human ear, and arranged to achieve electrical communication with said preselected contact areas in the conch of the right and left ears of a human subject when said respective protrusions are inserted therein.

2. The neuro-electric-therapy headset of claim 1, further comprising:
an audio source carried by said earpiece housings in juxtaposition to said tissue interface circuit, delivering audible signals responsive to impedance of said preselected contact area to the ear of said human subject during delivery of treatment signals.

3. The neuro-electric-therapy headset of claim 1, wherein said array of electrodes comprises four electrodes arranged approximately as quadrants of a circle.

4. The neuro-electric-therapy headset of claim 1, wherein:
said electrodes occupy substantially the entire area of said free end wall of each earpiece housing protrusion.

5. The neuro-electric-therapy headset of claim 1, further comprising:
right and left bow arms, each connected to said electronics housing and respectively carrying said right and left earpiece housings in positions placing said protrusions at a downward angle when applied to a human subject, suitable for simultaneously contacting said preselected contact areas in the conch of the respective right and left ears of the human subject.

6. The neuro-electric-therapy headset of claim 1, wherein:
said headset has front and rear faces coordinated to the front and rear of a human subject; and
further comprising right and left bow arms each connected to said electronics housing and respectively carrying said right and left earpiece housings in pivotable association such that the earpiece housings are pivotable with respect to the bow arms over a limited pivot range, from a neutral position wherein the earpieces face each other to a rearward angled position with respect to said rear face of the headset, whereby the earpiece housings are readily positioned for simultaneously inserting the respective right and left earpiece housings into the conch of the respective right and left ears of a human subject.

7. The neuro-electric-therapy headset of claim 6, wherein:
said pivot range is no more than thirty degrees rearward from said neutral position.

8. The neuro-electric-therapy headset of claim 2, wherein:
waveform source device is adapted to communicate selected waveforms to right and left tissue interface circuits while simultaneously measuring impedance at the right and left tissue interface circuits and generating a responsive audio signal through said audio source device.

9. The neuro-electric-therapy headset of claim 8, wherein:
said waveform source device delivers waveform signals suitable for trans-cranial treatment between an electrode associated with a first one of said right and left tissue interface circuits and an electrode associated with the second one of said right and left tissue interface circuits.

10. The neuro-electric-therapy headset of claim 8, wherein:
said waveform source device delivers waveform signals between at least two electrodes of said array of electrodes associated with a first one of said right and left tissue interface circuits.

11. The neuro-electric-therapy headset of claim 10, wherein:
said waveform source device simultaneously delivers waveform signals between at least two electrodes of said array of electrodes associated with a second one of said right and left tissue interface circuits.

12. A method of treating a human subject in need of such treatment for one or more disorders selected from chronic headache, migraine headache, hormonally induced migraine (PMS), narcotics withdrawal symptoms, smoking withdrawal symptoms, or any combination thereof, comprising:
providing a self-contained, portable headset carrying a selectively activated waveform source device with simultaneous impedance measuring means, carrying right and left tissue interface circuits responsive to said source device, and carrying an audio output means responsive to said impedance measuring means, wherein each of said right and left tissue interface circuits is configured with an ear-entering portion comprising an array of electrodes suitably sized and shaped for contacting the conch of a human ear at a preselected contact area near the lower edge of the ear canal opening and extending rearwardly there from;
applying said right and left tissue interface circuits respectively to right and left ears of a human subject, in a position such that said tissue interface circuit is in electrical communication with said preselected contact area;
activating said source device to provide waveform signals effective for treatment of said one or more disorders to both of said tissue interface circuits while simultaneously measuring impedance at the tissue interface circuits;
delivering effective waveform treatment over an effective time period for treating said condition to said preselected contact area in the conch of each ear of the human subject in need thereof, while simultaneously generating and delivering an audio output signal responsive to measured impedance at the tissue interface circuits for enabling the human subject to adjust the position of the headset for electrical communication with said preselected contact area.

13. The method of treatment according to claim 12, wherein each said array of electrodes comprises four electrodes arranged approximately as quadrants of a circle.

14. The method of treatment according to claim 13, wherein:
a first two electrodes of each said array are positive electrodes receiving positive waveform signals from said source device; and
a second two electrodes of each said array are negative electrodes;
whereby the source device delivers both ipsilateral and bilateral treatment.

15. The method of treatment according to claim 12, wherein said source device is cyclic in operation and provides treating frequencies in each cycle over a spectrum extending between two and two thousand hertz.
